(19) United States
(12) Patent Application Publication
(10) Pub. No.: US 2024/0130847 A1
(43) Pub. Date: Apr. 25, 2024

(54) PATELLA TENDON REALIGNMENT IMPLANT WITH CHANGEABLE SHAPE

(71) Applicant: ZKR ORTHOPEDICS, INC., San Francisco, CA (US)

(72) Inventors: Damien Shulock, San Francisco, CA (US); Jeffrey Halbrecht, San Francisco, CA (US); John Barrett, San Francisco, CA (US); Katherine J. Stephenson, San Francisco, CA (US)

(21) Appl. No.: 18/403,901

(22) Filed: Jan. 4, 2024

Related U.S. Application Data
(63) Continuation of application No. 17/127,634, filed on Dec. 18, 2020, now Pat. No. 11,896,476.
(60) Provisional application No. 62/956,562, filed on Jan. 2, 2020.

(51) Int. Cl. A61F 2/08

(52) U.S. Cl. CPC A61F 2/0811; A61F 2002/0835; A61F 2002/0858; A61F 2210/0014; A61F 2220/0091; A61F 2250/0003

(57) ABSTRACT

An orthopedic implant with an inferior portion having a tibia contact surface configured to extend over a tibia; a superior portion opposite to the inferior portion having a tendon contact surface configured to change a position of a patellar tendon by lifting or tilting the patellar tendon when the curved surface of the first portion is engaged with the tibia; and a fixation mechanism adapted to attach the orthopedic implant to the tibia, the orthopedic implant being further configured to change shape from a first configuration to a second configuration in response to a load applied between the tendon contact surface and the tibia contact surface. The invention also addresses corresponding methods.

### CROSS-REFERENCE TO RELATED APPLICATIONS

This application claims priority as a continuation of U.S. application Ser. No. 17/127,634, filed Dec. 18, 2020 and entitled “Patella Tendon Realignment Implant with Changeable Shape,” which claims the benefit under 35 U.S.C. § 119(e) to U.S. Provisional Application 62/956,562, filed Jan. 2, 2020 and entitled “Patella Tendon Realignment Implant with Changeable Shape,” both of which are hereby incorporated herein by reference in their entireties.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Problems of the patella-femoral joint are a common cause of knee pain. The pain may arise from issues such as poor alignment of the patella or from cartilage breakdown (chondromalacia or arthritis) behind the patella or on the opposing articular surface of the femoral groove (trochlea). Conventional surgical options for treating patella-femoral pain caused by malalignment, chondromalacia or arthritis may include realignment of the patella. For example tracking of the patella may be changed by tilting the patella or by moving the patella to one side or the other. Moving the patella forward (i.e., anteriorly) through a surgical procedure provides another conventional option for treating these conditions. This conventional technique is thought to decrease force across the patella-femoral joint and thus diminish the pain arising from excess force against a worn-out patellar or trochlear cartilage.

Although available, surgical options to realign the patella may be very invasive. For example, surgeries may involve cutting and fixating the bony attachment of the patellar tendon. In particular, conventional techniques may include detaching the patellar tendon from the tibia, then reattaching the patellar tendon at a new location to obtain the desired alignment of the patella. Such invasive surgical techniques may also result in prolonged recovery times. Consequently, an improved mechanism for treating patella-femoral joint problems such as patella-femoral pain, chondromalacia, and/or arthritis is desired.

U.S. Pat. No. 9,808,289 discloses embodiments of a patellar tendon realignment implant configured to be placed between the patellar tendon and the tibia in proximity to the patella to elevate and/or tilt the patellar tendon. Each of the implants described in the '289 patent maintains its shape despite changes to the load applied to it by the patellar tendon.

### SUMMARY OF THE DISCLOSURE

Some activities result in higher tension or compression of the patellar tendon. For example, stair climbing and jumping increase the tension on the patellar tendon, and kneeling or sudden impacts to the knee increase patellar tendon compression. It may be desirable to temporarily change the shape of a patellar tendon realignment implant during application of a tension load on the patellar tendon (e.g., during stair climbing, jumping, etc.) or application of a compression load on the patellar tendon (e.g., during kneeling, a sudden impact to the knee, etc.).

One aspect of the invention provides an orthopedic implant having an inferior portion having a tibia contact surface configured to extend over a tibia; a superior portion opposite to the inferior portion having a tendon contact surface configured to change a position of a patellar tendon by lifting or tilting the patellar tendon when the curved surface of the first portion is engaged with the tibia; and a fixation mechanism adapted to attach the orthopedic implant to the tibia, the orthopedic implant being further configured to change shape from a first configuration to a second configuration in response to a load applied between the tendon contact surface and the tibia contact surface.

In some embodiments, the distance between the tendon contact surface and the tibia contact surface is less in the second configuration than in the first configuration. For example, the distance between the tendon contact surface and the tibia contact surface may be up to 75% less, or ⅔ less, or 50% less in the second configuration than in the first configuration.

In some or all of these embodiments, the first configuration is an unloaded configuration. Alternatively, in some or all of these embodiments, the first configuration may be a partially loaded configuration.

In some or all of these embodiments, the superior portion may include a flexible material. In some such embodiments, the orthopedic implant may also have a first lateral side disposed between the inferior portion and the superior portion; and a second lateral side disposed between the inferior portion and the superior portion opposite to the first lateral side, wherein at least one of the first lateral side and the second lateral side comprises flexible material. In some other embodiments in which the orthopedic implant has a first lateral side disposed between the inferior portion and the superior portion; a second lateral side disposed between the inferior portion and the superior portion opposite to the first lateral side, the orthopedic implant may also have a central chamber defined by the superior portion, the inferior portion, the first lateral side and the second lateral side. In such embodiments, the central chamber comprises a gas or an elastic material.

In some embodiments, the orthopedic implant also has a cantilever connection between the superior portion and the inferior portion at an anterior side of the orthopedic implant, the superior portion being movable at the cantilever connection with respect to the inferior portion. In some such embodiments, the orthopedic implant has a posterior side opposite to the anterior side, the posterior side comprising an opening between the superior portion and the inferior portion, the opening having a height that changes as the superior portion moves at the cantilever connection with respect to the inferior portion. In other embodiments in which the orthopedic implant has a first lateral side and a second lateral side opposite to the first lateral side, the first lateral side and the second lateral side each have an opening between the superior portion and the inferior portion.

In some embodiments, the orthopedic implant includes deformable material. In some such embodiments, the superior portion has deformable material, and the inferior portion may also have a rigid material. In such embodiments, the fixation mechanism may include screw holes in the inferior portion. In some embodiments, the orthopedic implant also has a first lateral side disposed between the inferior portion and the superior portion and a second lateral side disposed between the inferior portion and the superior portion opposite to the first lateral side, and at least one of the first lateral portion and the second lateral portion has deformable material.

In some embodiments, the orthopedic implant has a spring operatively disposed between the superior portion and the inferior portion to permit the superior portion to move with respect to the inferior portion in response to a load applied between the tendon contact surface and the tibia contact surface. In some such embodiments, the spring is a cantilever spring connecting the superior portion to the inferior portion. For example, in some embodiments the inferior portion and the superior portion together comprise a wire spring form. In some such embodiments, the superior portion may have a roller. In other embodiments, the orthopedic implant may have a hinge connecting the superior portion to the inferior portion, and the spring may optionally be disposed between the superior portion and the inferior portion.

Yet another aspect of the invention provides a method for repositioning a patellar tendon. In some embodiments, the method includes the steps of: inserting an orthopedic implant in a first configuration between the patellar tendon and a tibia; engaging a tibia engagement surface of an inferior portion of the orthopedic implant with the tibia; engaging a patellar tendon surface of a superior portion of the orthopedic implant with the patellar tendon; changing a position of the patellar tendon; affixing the orthopedic implant to the tibia; and, in response to a load applied to the orthopedic implant by the patellar tendon, changing a shape of the orthopedic implant from the first configuration to a second configuration.

In some embodiments, the method includes the step of changing the shape of the orthopedic implant from the second configuration to the first configuration after removal of the load. In some or all of these embodiments, the step of changing a shape includes the step of reducing a distance between the tendon contact surface and the tibia contact surface. For example, the distance between the tendon contact surface and the tibia contact surface is up to 75% less in the second configuration than in the first configuration, or up to ⅔ less in the second configuration than in the first configuration, or up to 50% less in the second configuration than in the first configuration.

In some or all of these embodiments, the first configuration may be an unloaded configuration or a partially loaded configuration.

In some or all of these embodiments, the step of changing a shape may include the step of deforming at least a portion of the orthopedic implant. In some such embodiments, the step of deforming at least a portion of the orthopedic implant may include the step of deforming the superior portion. In some such embodiments in which the implant comprises a first lateral side disposed between the inferior portion and the superior portion and a second lateral side disposed between the inferior portion and the superior portion opposite to the first lateral side, the step of deforming at least a portion of the orthopedic implant may include the step of deforming at least one of the first lateral side and the second lateral side.

In some or all of these embodiments, the step of changing a shape may include the step of moving the patellar tendon surface and the tibia contact surface with respect to each other against a cantilever connection of the orthopedic implant.

In some or all of these embodiments, the step of changing a shape may include the step of moving the patellar tendon surface and the tibia contact surface with respect to each other against a spring of the orthopedic implant.

In some or all of these embodiments, the step of changing a shape may include the step of moving the patellar tendon surface and the tibia contact surface with respect to each other about a hinge of the orthopedic implant.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1A is a perspective view of an embodiment of a patellar implant.

FIG. 1B is a cross-sectional view of the patellar implant of FIG. 1A.

FIG. 2A is a perspective view of another embodiment of a patellar implant.

FIG. 2B is a cross-sectional view of the patellar implant of FIG. 2A.

FIG. 3A is a side view of yet another embodiment of a patellar implant.

FIG. 3B is a perspective view of the implant of FIG. 3A.

FIG. 4 is a perspective view of yet another embodiment of a patellar implant.

FIG. 5 is a perspective view of still another embodiment of a patellar implant.

FIG. 6 is a perspective view of another embodiment of a patellar implant.

FIGS. 7A and 7B show an implant according to embodiments of this invention in use on a patient.

### DETAILED DESCRIPTION

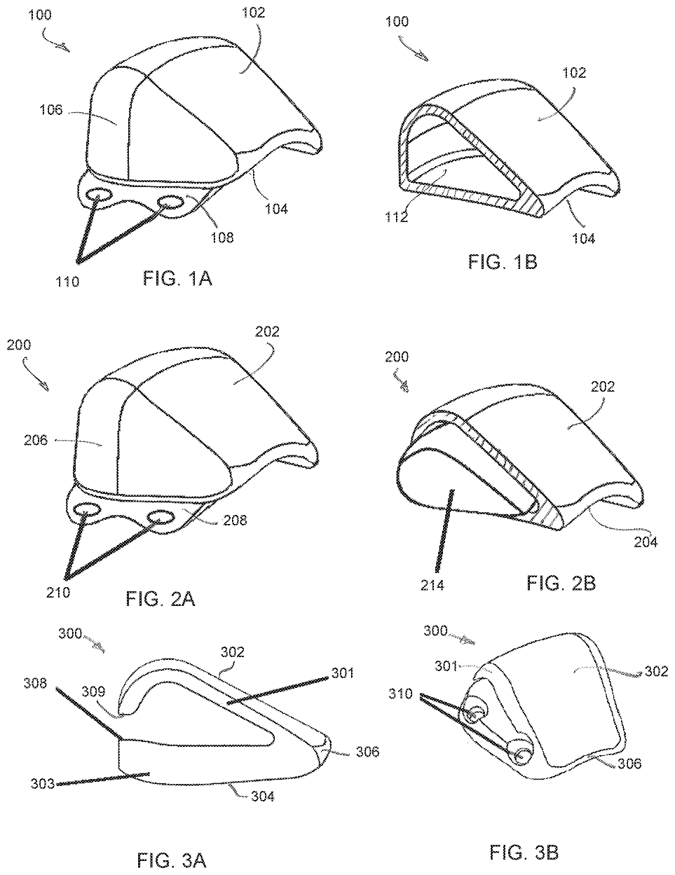

Implant 100 in FIGS. 1A and 1B has a superior portion with a tendon contact surface 102, an inferior portion with a tibia contact surface 104, a first lateral side 106 extending from the superior portion to the inferior portion, and a second lateral side (not shown) opposite to the first lateral side. A fixation tab 108 extends from the inferior portion on the first lateral side. Holes 110 in the fixation tab accommodate fixation screws or spikes (not shown). A similar fixation tab with holes extends from the inferior portion on the second lateral side as well.

Implant 100 may be implanted between a patient's patellar tendon and tibia to change the position of the patellar tendon by elevating it or lifting away from the tibia. When in place, the tibia contact surface 104 of the inferior portion is against, or in proximity to, the tibia. Tibia contact surface 104 may be curved to match the shape of the tibia. Alternatively, the tibia contact surface may be flat, and the tibia may be cut or shaved to accommodate the flat surface of the implant. When tibia contact surface 104 is in place on the tibia, tendon contact surface 102 will engage the patellar tendon to lift and/or tilt the tendon, thereby changing its position. Screws and/or spikes may be inserted through holes 110 into the tibia to hold the implant in place.

Implant 100 has a first configuration when it is placed on the tibia. The implant's shape and dimensions in this first configuration may be the same as its shape and dimensions prior to implant. Alternatively, the implant may be slightly compressed or deformed when first placed between the tibia and the patellar tendon.

After implantation, when the patient jumps or climbs stairs, tension on the patellar tendon will increase, thereby placing an increased load on implant 100. A different kind of load may be placed on implant 100 when the patient compresses the patellar tendon, such as by kneeling or encountering a sudden knee impact. Implant 100 responds to these loads by temporarily deforming to a second configuration. After the load is removed, implant 100 returns to the first configuration. This feature is provided by forming superior portion 102 and/or the lateral sides 106 of flexible material, such as rubber or plastic. In addition, a center chamber 112 surrounded in part by the superior portion 102, inferior portion 104, and the lateral sides 106 is filled with an inert gas to provide the ability to compress the implant to the second configuration and to return it to its first configuration. The flexible material's durometer and thickness, and the pressure of the inert gas inside of chamber 112, provide the necessary support for patellar tendon lift and/or tilt and as well as the ability for the implant to deform under a compressive force down to ⅓ of the implant's height in the first configuration and to return to the first configuration after removal of the compressive force. In other words, a distance between the tendon contact surface and the tibia contact surface in the second configuration is up to ⅔ less than in the first configuration.

The embodiment of FIGS. 2A and 2B is similar to that of FIGS. 1A and 1B. Implant 200 may be implanted between a patient's patellar tendon and tibia to change the position of the patellar tendon by elevating it or lifting away from the tibia. When in place, tibia contact surface 204 of the inferior portion is against, or in proximity to, the tibia. Tibia contact surface 204 may be curved to match the shape of the tibia. Alternatively, the tibia contact surface may be flat, and the tibia may be cut or shaved to accommodate the flat surface of the implant. When tibia contact surface 204 is in place on the tibia, tendon contact surface 202 will engage the patellar tendon to lift and/or tilt the tendon, thereby changing its position. Screws and/or spikes may be inserted through holes 210 into the tibia to hold the implant in place.

Implant 200 has a first configuration when it is placed on the tibia. The implant's shape and dimensions in this first configuration may be the same as its shape and dimensions prior to implant. Alternatively, the implant may be slightly compressed or deformed when first placed between the tibia and the patellar tendon.

After implantation, when the patient jumps or climbs stairs, tension on the patellar tendon will increase, thereby placing an increased load on implant 200. A different kind of load may be placed on implant 200 when the patient compresses the patellar tendon, such as by kneeling or encountering a sudden knee impact. Implant 200 responds to these loads by temporarily deforming to a second configuration. After the load is removed, implant 200 returns to the first configuration. This feature is provided by forming superior portion 202 and/or the lateral sides 206 of flexible material, such as rubber or plastic. In addition, a center chamber 212 surrounded in part by the superior portion 202, inferior portion 204, and the lateral sides 206 is filled with a core 214 formed from an elastic material to provide the ability to compress the implant to the second configuration and to return it to its first configuration. The flexible material's durometer and thickness, and the elastic properties of core 214, provide the necessary support for patellar tendon lift and/or tilt and as well as the ability for the implant to deform under a compressive force down to ⅓ of the implant's height in the first configuration and to return to the first configuration after removal of the compressive force. In other words, a distance between the tendon contact surface and the tibia contact surface in the second configuration is up to ⅔ less than in the first configuration.

FIGS. 3A and 3B show yet another embodiment of a deformable patellar implant. Implant 300 has a superior portion 301 with a tendon contact surface 302 joined at a cantilever portion 306 on the implant's anterior side to an inferior portion 303 having a tibia contact surface 304. As in the other embodiments, implant 300 may be implanted between a patient's patellar tendon and tibia to change the position of the patellar tendon by elevating it or lifting away from the tibia. When in place, tibia contact surface 304 of the inferior portion is against, or in proximity to, the tibia. Tibia contact surface 304 may be curved to match the shape of the tibia. Alternatively, the tibia contact surface may be flat, and the tibia may be cut or shaved to accommodate the flat surface of the implant. When tibia contact surface 304 is in place on the tibia, tendon contact surface 302 will engage the patellar tendon to lift and/or tilt the tendon, thereby changing its position. Screws and/or spikes may be inserted through holes 310 into the tibia to hold the implant in place.

Implant 300 has a first configuration when it is placed on the tibia. The implant's shape and dimensions in this first configuration may be the same as its shape and dimensions prior to implant. Alternatively, the implant may be slightly compressed or deformed when first placed between the tibia and the patellar tendon.

After implantation, when the patient jumps or climbs stairs, tension on the patellar tendon will increase, thereby placing an increased load on implant 300. A different kind of load may be placed on implant 300 when the patient compresses the patellar tendon, such as by kneeling or encountering a sudden knee impact. Implant 300 responds to these loads by temporarily deforming to a second configuration by flexing superior portion 301 with respect to inferior portion 303 about the cantilever portion 306. As it deforms, a gap 308 on the posterior side of implant 300 gets smaller and possibly closes altogether as surface 309 meets the top of inferior portion 301. After the load is removed, implant 300 returns to the first configuration. Implant 300 may be made of metal, plastic or a combination thereof so that it can sustain multiple flexing cycles without fatigue and breakage. The flexible material's elastic modulus and geometry are calibrated to provide the necessary support required for tendon lift and/or redirection and the necessary deformation under compressive forces.

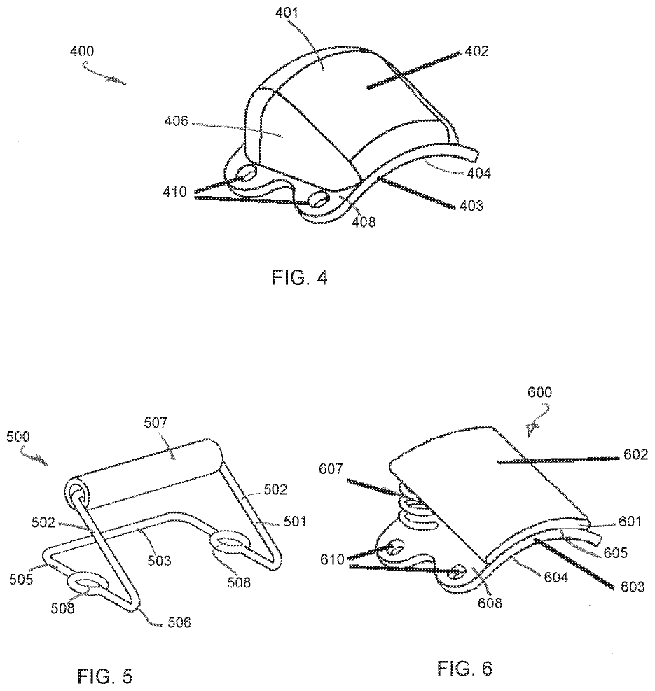

FIG. 4 shows another embodiment of a deformable patellar implant according to the invention. Implant 400 has an inferior portion 403 with a tibia contact surface 404, a superior portion 401 with a tendon contact surface 402, a first lateral side 406 extending from the superior portion to the inferior portion, and a second lateral side (not shown) opposite to the first lateral side. A fixation tab 408 extends from the inferior portion on the first lateral side. Holes 410 in the fixation tab accommodate fixation screws or spikes (not shown). A similar fixation tab with holes extends from the inferior portion on the second lateral side as well.

Implant 400 may be implanted between a patient's patellar tendon and tibia to change the position of the patellar tendon by elevating it or lifting away from the tibia. When in place, the tibia contact surface 404 of the inferior portion is against, or in proximity to, the tibia. Tibia contact surface 404 may be curved to match the shape of the tibia. Alternatively, the tibia contact surface may be flat, and the tibia may be cut or shaved to accommodate the flat surface of the implant. When tibia contact surface 404 is in place on the tibia, tendon contact surface 402 will engage the patellar tendon to lift and/or tilt the tendon, thereby changing its position. Screws and/or spikes may be inserted through holes 410 into the tibia to hold the implant in place.

Implant 400 has a first configuration when it is placed on the tibia. The implant's shape and dimensions in this first configuration may be the same as its shape and dimensions prior to implant. Alternatively, the implant may be slightly compressed or deformed when first placed between the tibia and the patellar tendon.

After implantation, when the patient jumps or climbs stairs, tension on the patellar tendon will increase, thereby placing an increased load on implant 400. A different kind of load may be placed on implant 400 when the patient compresses the patellar tendon, such as by kneeling or encountering a sudden knee impact. Implant 400 responds to these loads by temporarily deforming to a second configuration. After the load is removed, implant 400 returns to the first configuration. This feature is provided by forming the superior portion 401 and/or the lateral sides 406 of soft and deformable material, such as silicone rubber. Inferior portion 403, on the other hand, is rigid plastic or metal. The deformable portions (superior portion 401 and/or lateral sides 406) have a durometer and geometry calibrated to provide the support needed for tendon lift and/or redirection, and under compressive forces can deform down to ½ of the implant's original height in the first configuration and to return to the first configuration after removal of the compressive force. In other words, a distance between the tendon contact surface and the tibia contact surface in the second configuration is up to 50% less than in the first configuration.

FIG. 5 shows still another embodiment of a deformable patellar implant. Implant 500 has a wire spring form 501 made of, e.g., spring-tempered stainless steel or titanium. Spring form 501 extends from an inferior posterior portion 503 and two inferior side portions 505 to cantilever sections 506. The underside of inferior posterior portion 503 and the inferior side portions 505 together form a tibia contact surface. Two superior side portions 502 extend upwards from cantilever sections 506 to a posterior roller 507, which provides a tendon contact surface. Screw mounts 508 extend from inferior side portions 505 and can accommodate fixation screws or spikes.

Implant 500 may be implanted between a patient's patellar tendon and tibia to change the position of the patellar tendon by elevating it or lifting away from the tibia. When in place, the tibia contact surface is against, or in proximity to, the tibia. When the tibia contact surface is in place on the tibia, roller 507 will engage the patellar tendon to lift and/or tilt the tendon, thereby changing its position. Screws and/or spikes may be inserted through holes 508 into the tibia to hold the implant in place.

Implant 500 has a first configuration when it is placed on the tibia. The implant's shape and dimensions in this first configuration may be the same as its shape and dimensions prior to implant. Alternatively, the implant may be slightly compressed or deformed when first placed between the tibia and the patellar tendon.

After implantation, when the patient jumps or climbs stairs, tension on the patellar tendon will increase, thereby placing an increased load on implant 500. A different kind of load may be placed on implant 500 when the patient compresses the patellar tendon, such as by kneeling or encountering a sudden knee impact. Implant 500 responds to these loads by temporarily deforming to a second configuration. After the load is removed, implant 500 returns to the first configuration. The spring form material, temper condition, and geometry are calibrated together to provide the support needed to lift and/or redirect the patellar tendon. Under compressive forces, the implant 500 can deform down to ¼ of the implant's original height in the first configuration and to return to the first configuration after removal of the compressive force. In other words, a distance between the tendon contact surface and the tibia contact surface in the second configuration is up to 75% less than in the first configuration.

FIG. 6 shows an embodiment of a patellar implant in which a superior portion 601 connects to an inferior portion 603 via a hinge 605 and a spring 607. Superior portion 601 has a tendon contact surface 602, and inferior portion 603 has a tibia contact surface 604. A fixation tab 608 extends from the inferior portion on both lateral sides. Holes 610 in the fixation tab accommodate fixation screws or spikes (not shown). The superior portion 601 and inferior portion 603 may be made from rigid plastic or metal.

Implant 600 may be may be implanted between a patient's patellar tendon and tibia to change the position of the patellar tendon by elevating it or lifting away from the tibia. When in place, the tibia contact surface 604 of the inferior portion is against, or in proximity to, the tibia. Tibia contact surface 604 may be curved to match the shape of the tibia. Alternatively, the tibia contact surface may be flat, and the tibia may be cut or shaved to accommodate the flat surface of the implant. When tibia contact surface 604 is in place on the tibia, tendon contact surface 602 will engage the patellar tendon to lift and/or tilt the tendon, thereby changing its position. Screws and/or spikes may be inserted through holes 610 into the tibia to hold the implant in place.

Implant 600 has a first configuration when it is placed on the tibia. The implant's shape and dimensions in this first configuration may be the same as its shape and dimensions prior to implant. Alternatively, the implant may be slightly compressed or deformed when first placed between the tibia and the patellar tendon.

After implantation, when the patient jumps or climbs stairs, tension on the patellar tendon will increase, thereby placing an increased load on implant 600. A different kind of load may be placed on implant 600 when the patient compresses the patellar tendon, such as by kneeling or encountering a sudden knee impact. Implant 600 responds to these loads by moving superior portion 601 about hinge 605 away from inferior portion 603 and against the action of spring 607 to temporarily change the implant's shape to a second configuration. After the load is removed, spring 607 returns implant 600 to the first configuration. The spring element material, temper condition, and geometry are calibrated together to provide the necessary support for tendon lift and/or redirection as well as the ability for the implant to change shape under a compressive force down to ⅓ of the implant's height in the first configuration and to return to the first configuration after removal of the compressive force. In other words, a distance between the tendon contact surface and the tibia contact surface in the second configuration is up to ⅔ less than in the first configuration.

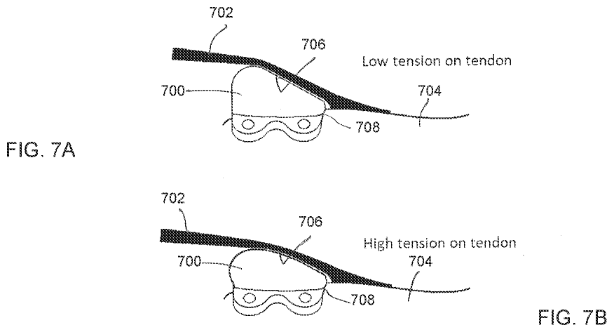

FIGS. 7A and 7B demonstrate use of an implant according to embodiments of this invention in use on a patient. Implant 700 (such as, e.g., an implant shown in FIGS. 1, FIG. 2 or FIG. 4) is affixed to a tibia 704 of the patient. FIG. 7A shows the shape and configuration of the implant 700 when patellar tendon 702 is under little or no tension. FIG. 7B shows the shape and configuration of implant 700 when tendon 702 is under high tension. When under tension, tendon 702 exerts a load on implant 700 and compresses the implant to reduce the distance from the tendon contact surface 706 and the tibia contact surface 708 as shown in FIG. 7B.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. An orthopedic implant comprising:
(a) an inferior portion having a tibia contact surface configured to extend over a tibia;
(b) a superior portion opposite to the inferior portion having a tendon contact surface configured to change a position of a patellar tendon by lifting or tilting the patellar tendon when the curved surface of the first portion is engaged with the tibia; and
(c) a fixation mechanism adapted to attach the orthopedic implant to the tibia,
the orthopedic implant being further configured to change shape from a first configuration to a second configuration in response to a load applied between the tendon contact surface and the tibia contact surface.

2. The orthopedic implant of claim 1 wherein a distance between the tendon contact surface and the tibia contact surface is less in the second configuration than in the first configuration.

3. The orthopedic implant of claim 2 wherein the distance between the tendon contact surface and the tibia contact surface is up to 75% less in the second configuration than in the first configuration.

4. The orthopedic implant of claim 2 wherein the distance between the tendon contact surface and the tibia contact surface is up to ⅔ less in the second configuration than in the first configuration.

5. The orthopedic implant of claim 2 wherein the distance between the tendon contact surface and the tibia contact surface is up to 50% less in the second configuration than in the first configuration.

6. The orthopedic implant of claim 1 wherein the first configuration is an unloaded configuration.

7. The orthopedic implant of claim 1 wherein the first configuration is a partially loaded configuration.

8. The orthopedic implant of claim 1 wherein the superior portion comprises flexible material.

9. The orthopedic implant of claim 8 further comprising:
(a) a first lateral side disposed between the inferior portion and the superior portion; and
(b) a second lateral side disposed between the inferior portion and the superior portion opposite to the first lateral side,
wherein at least one of the first lateral side and the second lateral side comprises flexible material.

10. The orthopedic implant of claim 8 further comprising:
(a) a first lateral side disposed between the inferior portion and the superior portion;
(b) a second lateral side disposed between the inferior portion and the superior portion opposite to the first lateral side; and
(c) a central chamber defined by the superior portion, the inferior portion, the first lateral side and the second lateral side.

11. The orthopedic implant of claim 10 wherein the central chamber comprises a gas.

12. The orthopedic implant of claim 10 wherein the central chamber comprises an elastic material.

13. The orthopedic implant of claim 1 further comprising a cantilever connection between the superior portion and the inferior portion at an anterior side of the orthopedic implant, the superior portion being movable at the cantilever connection with respect to the inferior portion.

14. The orthopedic implant of claim 13 further comprising a posterior side opposite to the anterior side, the posterior side comprising an opening between the superior portion and the inferior portion, the opening having a height that changes as the superior portion moves at the cantilever connection with respect to the inferior portion.

15. The orthopedic implant of claim 13 further comprising a first lateral side and a second lateral side opposite to the first lateral side, the first lateral side and the second lateral side each comprising an opening between the superior portion and the inferior portion.

16. The orthopedic implant of claim 1 further comprising deformable material.

17. The orthopedic implant of claim 16 wherein the superior portion comprises deformable material.

18. The orthopedic implant of claim 17 wherein the inferior portion comprises rigid material.

19. The orthopedic implant of claim 18 wherein the fixation mechanism comprises screw holes in the inferior portion.

20. The orthopedic implant of claim 16 further comprising a first lateral side disposed between the inferior portion and the superior portion and a second lateral side disposed between the inferior portion and the superior portion opposite to the first lateral side, at least one of the first lateral portion and the second lateral portion comprises deformable material.

21. The orthopedic implant of claim 1 further comprising a spring operatively disposed between the superior portion and the inferior portion to permit the superior portion to move with respect to the inferior portion in response to a load applied between the tendon contact surface and the tibia contact surface.

22. The orthopedic implant of claim 21 wherein the spring comprises a cantilever spring connecting the superior portion to the inferior portion.

23. The orthopedic implant of claim 22 wherein the inferior portion and the superior portion together comprise a wire spring form.

24. The orthopedic implant of claim 23 wherein the superior portion comprises a roller.

25. The orthopedic implant of claim 21 further comprising a hinge connecting the superior portion to the inferior portion.

26. The orthopedic implant of claim 25 wherein the spring is disposed between the superior portion and the inferior portion.
